(19) United States
(12) Patent Application Publication
(10) Pub. No.: US 2026/0242389 A1
(43) Pub. Date: Aug. 20, 2026

(54) MULTI-FUNCTIONAL RHODAMINE 6G DERIVATIVE ATTACHED WITH 2-THIAZOLYLETHYL AMINE AND PREPARATION METHOD AND USE THEREOF

(71) Applicant: SOOCHOW UNIVERSITY, Suzhou (CN)

(72) Inventors: Dongmei XU, Suzhou (CN); Zhiyi Li, Suzhou (CN)

(21) Appl. No.: 19/422,868

(22) Filed: Dec. 17, 2025

(30) Foreign Application Priority Data
Feb. 17, 2025 (CN) 202510168311.7

(51) Int. Cl. C07D 491/107; G01N 21/33; G01N 21/64

(52) U.S. Cl. CPC C07D 491/107; G01N 21/33; G01N 21/64

(57) ABSTRACT

A multi-functional rhodamine 6G derivative attached with 2-thiazolylethyl amine, a preparation method and use thereof are provided. The multi-functional rhodamine 6G derivative attached with 2-thiazolylethyl amine is prepared by a one-step method with rhodamine 6G and 2-thiazolylethyl amine as raw materials. In the present invention, a new multi-functional rhodamine 6G derivative RGET is synthesized by a one-step method. RGET is useful as a highly selective and sensitive Fe³⁺ spectroscopic probe, the detection limit for Fe³⁺ is 2.1 nM, and the fluorescence intensity is increased by 541 times by Fe³⁺. Moreover, RGET has antibacterial performances comparable to those of commercial bleach for Gram-positive bacteria and Gram-negative bacteria and is very lowly cytotoxic, thus having bright application prospects in the fields of environment protection and life sciences.

This application claims priority to Chinese Patent Application No. 202510168311.7, filed on Feb. 17, 2025, which is incorporated by reference for all purposes as if fully set forth herein.

### FIELD OF THE INVENTION

The present invention relates to the technical field of advanced materials, and concerns a functional rhodamine derivative and specifically a multi-functional rhodamine 6G derivative attached with 2-thiazolylethyl amine and preparation method and use thereof.

### DESCRIPTION OF THE RELATED ART

Rhodamine derivatives have long absorption and emission wavelengths, high fluorescence quantum yield, unique open-closed loop structure, and corresponding absorption and emission spectral changes, color and fluorescence changes. Therefore, as a spectroscopic probe, they are widely used in many fields, such as heavy metal ion detection, fluorescence labeling, and living cell imaging (see: Fluorogenic and cell permeable rhodamine dyes for high contrast live cell protein labeling in bioimaging and biosensing).

Fe³⁺ is widely present in human tissues and organs, and involved in various life activities such as cell metabolism and DNA or protein synthesis (see: Two to tango: regulation of Mammalian iron metabolism), so it is very important to know the content of Fe³⁺ in the environment and within living organisms. Fe³⁺ spectroscopic probe method is a Fe³⁺ detection method receiving great attention in recent years, and rhodamine derivatives are very popular spectroscopic probes. For example, Dipankar Das et al. reported a rhodamine 6G-based Fe³⁺ spectroscopic probe in 2024. In a 10 mM HEPES aqueous solution pH 7.2, the fluorescence intensity of the probe is increased by 14 times by Fe³⁺, and there is a linear relationship between the fluorescence intensity of the probe and Fe³⁺ concentration in the range of 30-60 mM. The detection limit of Fe³⁺ is 4.2 μM (see: A rhodamine-6G based chromo and fluorogenic sensor for the selective turn-ON detection of Fe³⁺ in aqueous medium with potential applications as INHIBITlogic gate, memory device and live cell imaging). For example, Chen et al. reported a rhodamine B derivative-based Fe³⁺ fluorescent probe in 2017. In a solution with MeOH/H₂O=1/2 (volume ratio, pH 7.4, Tris-HCl buffer, 1 mM), the fluorescence intensity of the probe is increased by 21 times by Fe³⁺ and there is a linear relationship between the fluorescence intensity and Fe³⁺ concentration in a range of Fe³⁺ concentration of 0-20 μM. The detection limit of Fe³⁺ is 11.6 nM. Many similar reports are available, but most of them only show a single function of detecting Fe³⁺ through spectroscopic changes.

If the rhodamine derivative can have multiple functions at the same time, it is of greater significance compared to the situation where only one metal ion is detected. Therefore, the research of multifunctional rhodamine derivatives has attracted more and more attention. At present, the reported multifunctional spectroscopic probes can only be used to identify multiple ions or molecules, exhibiting functions belonging to the same field, and rhodamine derivatives with multi-functions belonging to different fields have not been reported.

### SUMMARY OF THE INVENTION

In the present invention, a new multi-functional rhodamine 6G derivative RGET is synthesized by a one-step method. RGET can be used as a highly selective and sensitive Fe³⁺ spectroscopic probe, where the detection limit of Fe³⁺ is 2.1 nM, and the fluorescence intensity is increased by 541 times by Fe³⁺. Moreover, RGET has antibacterial properties comparable to those of commercial bleach for Gram-positive bacteria and Gram-negative bacteria, and is lowly cytotoxic, so it has bright application prospects in the fields of environment protection and life sciences.

The following technical solutions are adopted in the present invention.

A multi-functional rhodamine 6G derivative attached with 2-thiazolylethyl amine has a chemical structural formula below:

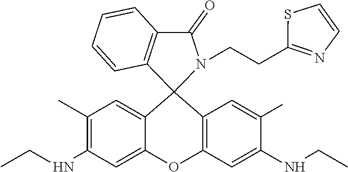 [SMILES: CCNc1cc2c(cc1C)C1(c3cc(C)c(NCC)cc3O2)c2ccccc2C(=O)N1CCc1nccs1]

The present invention discloses a method for preparing the multi-functional rhodamine 6G derivative attached with 2-thiazolylethyl amine, which includes the following step: preparing the multi-functional rhodamine 6G derivative attached with 2-thiazolylethyl amine by a one-step reaction using rhodamine 6G and 2-thiazolylethyl amine as raw materials.

In the present invention, the reaction is carried out under an inert gas atmosphere. Preferably, the inert gas atmosphere includes nitrogen atmosphere.

In the present invention, the reaction temperature is 50 to 100° C., and the time is 1 to 15 h. Preferably, the reaction temperature is 65 to 85° C., and the time is 2 to 10 h.

The present invention discloses use of the multi-functional rhodamine 6G derivative attached with 2-thiazolylethyl amine as a Fe³⁺ spectroscopic probe.

The present invention discloses use of the multi-functional rhodamine 6G derivative attached with 2-thiazolylethyl amine in the preparation of a Fe³⁺ detection reagent.

The present invention discloses use of the multi-functional rhodamine 6G derivative attached with 2-thiazolylethyl amine as an antibacterial agent.

The present invention discloses use of the multi-functional rhodamine 6G derivative attached with 2-thiazolylethyl amine in the preparation of an antibacterial agent.

The present invention discloses a method for detecting Fe³⁺, which includes the following steps: detecting Fe³⁺ with the multi-functional rhodamine 6G derivative attached with 2-thiazolylethyl amine. Particularly, the method includes the following steps: mixing the multi-functional rhodamine 6G derivative attached with 2-thiazolylethyl amine with a to-be-detected system, and detecting Fe³⁺ spectroscopically.

In the present invention, the spectroscopic detection includes one or both of ultraviolet-visible spectroscopic detection and fluorescent spectroscopic detection.

In the present invention, the solvent system for spectroscopic detection comprises an organic solvent and water, where the organic solvent includes acetonitrile, N,N-dimethyl formamide (DMF), and others, and preferably DMF.

Preferably, the volume ratio of the organic solvent to water is 1:(50-150). Further preferably, the volume ratio of the organic solvent to water is 1:(70-120). Further preferably, the volume ratio of the organic solvent to water is 1:(90-110).

The present invention provides an antibacterial method, which includes the following steps: combating bacteria with the multi-functional rhodamine 6G derivative attached with 2-thiazolylethyl amine. Particularly, the method includes the following steps: mixing the multi-functional rhodamine 6G derivative attached with 2-thiazolylethyl amine with a bacterium containing system, to combat the bacteria.

In the present invention, the bacteria include one or more of Gram-positive bacteria and Gram-negative bacteria.

Particularly, the Gram-positive bacteria include one of more of Staphylococcus, Enterococcus, Streptococcus, Bacillus, Corynebacterium, and the like; and the Gram-negative bacteria include one or more of Klebsiella, Escherichia coli, Pseudomonas aeruginosa, Haemophilus influenzae, Salmonella and the like. For example, the antibacterial performances of RGET of the present invention against Bacillus thuringiensis and Serratia marcescens are comparable to those of commercial bleach, and the cytotoxicity is very low, showing a good application prospect in the fields of environmental monitoring and biomedicines.

In the present invention, a new rhodamine 6G derivative is designed and synthesized, which is a multi-functional rhodamine 6G derivative useful as a Fe³⁺ spectroscopic probe and having antibacterial performance. Compared with the prior art, the present invention has the following positive effect and advantages:
1. RGET is simple to synthesize;
2. RGET has multiple functions in different fields, including Fe³⁺ identification and antibacterial performance;
3. RGET, as a Fe³⁺ spectroscopic probe, has good selectivity and high sensitivity;
4. RGET has almost no cytotoxicity; and
5. RGET has antibacterial properties close to those of commercial bleach.

### BRIEF DESCRIPTION OF THE DRAWINGS

FIG. 1 shows a schematic synthesis scheme in the present invention.

FIG. 2 shows photos showing the response of RGET (10 μM) to Fe³⁺, Al³⁺, and Cr³⁺ in a CH₃CN/H₂O (10/90, 20/80, 30/70, 40/60, 50/50, 60/40, 70/30, 80/20 and 90/10, v/v) solvent system, where upper: natural light, and lower: UV at 365 nm; and the metal concentration is 50 μM.

FIG. 3 shows photos showing the response of RGET (10 μM) to 17 metal ions in a DMF/H₂O (1/99, v/v) solvent system, where upper: natural light, and lower: UV at 365 nm; and the metal concentration is 100 μM.

FIG. 4 shows the selectivity of RGET to metal ions, where a is a UV-vis absorption spectrum, and b is a fluorescent spectrum; solvent: DMF/H₂O (1/99, v/v); RGET concentration: 10 μM; metal ion concentration: 100 μM; excitation wavelength: 530 nm; slit width: 2.5 nm.

FIG. 5 shows the spectrum of RGET in the presence of various concentrations of Fe³⁺, where a is a UV-vis absorption spectrum, and b is a fluorescent spectrum, illustrations: the relationship between the concentration of Fe³⁺ and the absorbance at 531 nm (a)/the fluorescence intensity at 554 nm (b); solvent: DMF/H₂O (1/99, v/v); RGET concentration: 10 μM; metal ion concentration: 0-150 M; maximum absorption wavelength: 531 nm, excitation wavelength: 530 nm, slit width: 2.5 nm, fluorescence wavelength: 554 nm.

FIG. 6 shows the effect of coexisting metal ions on the solution of RGET-Fe³⁺, where a shows the absorbance at 531 nm and b shows the fluorescence intensity at 554 nm; concentration: 10 μM for RGET, 100 μM for Fe³⁺, and 100 μM for other ions; solvent system: DMF/H₂O (1/99, v/v); maximum absorption and excitation wavelengths: 530 nm, slit width: 2.5 nm; fluorescence wavelength: 554 nm.

FIG. 7 shows the time response of RGET-Fe³⁺ solution, where a shows the UV-vis absorbance, and b shows the fluorescence intensity; concentration: 10 μM for RGET, and 100 μM for Fe³⁺; solvent system: DMF/H₂O (1/99, v/v); maximum absorption and excitation wavelengths: 530 nm, slit width: 2.5 nm, fluorescence wavelength: 554 nm.

FIG. 8 shows the reversibility of RGET in the detection of Fe³⁺, where a is a UV-vis absorption spectrum, and b is a fluorescent spectrum; concentration: 10 μM for RGET, 100 μM for Fe³⁺, and 110 μM for EDTA; solvent: DMF/H₂O (1/99, v/v); maximum absorption and excitation wavelengths: 530 nm; slit width: 2.5 nm, fluorescence wavelength: 554 nm; illustrations: the color of an RGET solution in DMF/H₂O (1/99, v/v) under natural light after Fe³⁺ is added and then excess EDTA solution is added and the fluorescence change under UV at 365 nm; upper: natural light, and lower: UV at 365 nm.

FIG. 9 shows the survival rate of CIK cells incubated with RGET of 20, 40, and 60 μM.

FIG. 10 shows photos of quantitative killing experiments with RGET in suspension, where a: Bacillus thuringiensis+sterilized water; b: Bacillus thuringiensis+DMSO; c, d, e, f, and g: Bacillus thuringiensis+RGET solution (having a concentration of 2.0, 3.0, 3.1, 3.2, and 3.3 mg/mL respectively); h: Bacillus thuringiensis+bleach, where the concentration of bleach is 2.0 mg/mL.

FIG. 11 shows photos of quantitative killing experiments with RGET in suspension, where a: Serratia marcescens+sterilized water; b: Serratia marcescens+DMSO; c, d, e, f, and g: Serratia marcescens+RGET solution (having a concentration of 2.0, 3.0, 3.1, 3.2, and 3.3 mg/mL respectively); h: Serratia marcescens+bleach, where the concentration of bleach is 2.0 mg/mL.

FIG. 12 shows photos showing the response of RGTM (a), RGET (b) and RGAT (c) (10 μM) to 17 metal ions (50 μM) in a DMF/H₂O (1/99, v/v) system, where gray background: natural light, and dark blue background: UV at 365 nm.

### DETAILED DESCRIPTION OF THE PREFERRED EMBODIMENTS

The present invention discloses the chemical structure of a multi-functional rhodamine 6G derivative attached with 2-thiazolylethyl amine and a synthesis method thereof, also discloses use of the rhodamine 6G derivative in Fe³⁺ spectroscopic probes, and further discloses use of the rhodamine 6G derivative in bacterium inhibition.

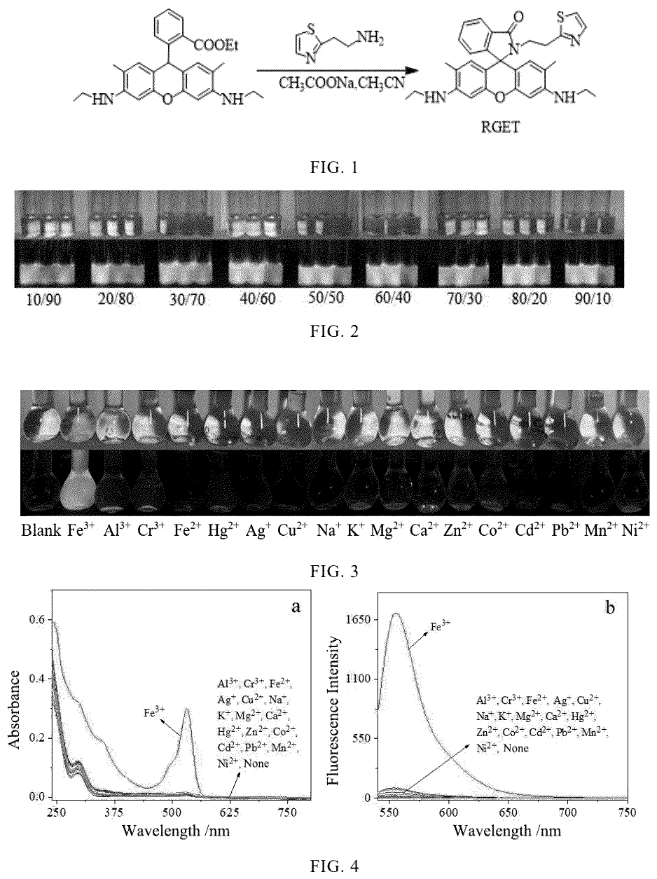

Referring to FIG. 1, in the present invention, rhodamine 6G and 2-thiazolylethyl amine are used as raw materials and reacted to prepare the multi-functional rhodamine 6G derivative attached with 2-thiazolylethyl amine. Specifically: rhodamine 6G and sodium acetate (or N,N-diisopropyl ethyl amine) are dissolved in a solvent. 2-thiazolylethyl amine is added under a N₂ atmosphere, and reacted under reflux. After the reaction, the reaction solution is cooled to room temperature, the solvent is removed under reduced pressure, and the crude product is separated by column chromatography on silica gel to obtain a product as particles, yield 26.1%-75.2%.

In the present invention, the molar ratio of rhodamine 6G to 2-thiazolylethyl amine is 1:(0.5-3), and preferably, the molar ratio of rhodamine 6G to 2-thiazolylethyl amine is 1:(1-2.5).

In the present invention, the time for reaction under reflux is 3-6 h, and the temperature is 65-85° C.

In the present invention, the molar ratio of rhodamine 6G to sodium acetate is 1:(1-3), and preferably, the molar ratio of rhodamine 6G to sodium acetate is 1:(1.5-2.5).

In the present invention, the molar ratio of rhodamine 6G to N,N-diisopropyl ethyl amine is 1:(1-3), and preferably, the molar ratio of rhodamine 6G to N,N-diisopropyl ethyl amine is 1:(1.5-2.5).

In the present invention, the solvent includes acetonitrile, and ethanol, etc.

Hereinafter, the technical progress of the present invention is illustrated by specific experiments, in which the raw materials (cells and bacteria) used are all commercially available products, and the specific preparation operation and performance test are conventional technologies. The preparation of metal ions is a conventional in the art, which does not affect the understanding of the technical effect of the present invention by technicians in the art. Corresponding compounds used in the preparation of metal ion solutions of Fe³⁺, Fe²⁺, Cr³⁺, Ag⁺, Cu²⁺, Al³⁺, Na⁺, K⁺, Mg²⁺, Ca²⁺, Hg²⁺, Zn²⁺, Co²⁺, Cd²⁺, Pb²⁺, Mn²⁺, and Ni²⁺: anhydrous FeCl₃, FeCl₂·7H₂O, CrCl₃·6H₂O, AgNO₃, CuSO₄·5H₂O, AlCl₃·6H₂O, NaCl, KCl, MgCl₂, CaCl₂, HgCl₂, Zn(NO₃)₂·6H₂O, CoCl₂·6H₂O, CdCl₂·2.5H₂O, Pb(NO₃)₂, MnSO₄·H₂O, and Ni(NO₃)₂·6H₂O.

### Example 1: Synthesis of RGET

### (1) Synthesis Method:

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (11 μL, 0.1 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 19.7 mg of a product as light orange particles (yield 75.2%).

### (2) Structural Characterization of RGET:

RGET has a molecular formula of C₃₁H₃₂N₄O₂S and a molecular weight M of 524.22. The peak with a mass-to-charge ratio of 525.2187 was found to be the [M+H]⁺ molecular ion peak of RGET by MS. Then the sample was analyzed by IR spectroscopy, and the characteristic absorption peak (v/cm⁻¹) of the RGAT structure was observed: v (C—O—C): 1268. v (Ar—H): 1402, 1503, 713, v (C—N): 1345, v (C═O): 1616, v (C═N): 1688, v (C—S—C): 1034, v (CH₃, CH₂): 2960, 2914. The sample was further analyzed by 1H NMR, and the following data was obtained: 1H NMR (400 MHz, CDCl₃, δ/ppm): 7.94 (dd, J=5.9, 1H, PhH), 7.55 (dd, J=5.8, 1H, PhH), 7.08-7.13 (t, J=3.5 Hz, 2H, PhH, SCHCHN), 7.01-7.05 (d, J=3.3 Hz, 3H, PhH)), 7.44 (m, 1H, SCHCHN), 3.50-3.59 (m, J=3.2 Hz, 2H, CH₂CH₂N), 2.84-2.93 (m, 2H, CH₂CH₂N), 6.25 (s, 2H, PhH), 6.35 (s, 2H, CH₃CH₂NH), 3.17-3.23 (m, 4H, CH₃CH₂NH), 1.87 (s, 6H, PhCH₃), 1.30 (t, J=7.2 Hz, 6H, CH₃CH₂NH). Based on the above analysis results, the sample is RGET.

### Example 2: Synthesis of RGET

Rhodamine 6G (0.024 g, 0.05 mmol) and sodium acetate (CH₃COONa) (0.0082 g, 0.1 mmol) were dissolved in ethanol (8 mL). Under a N₂ atmosphere, 2-thiazolylethyl amine (8 μL, 0.075 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 6.8 mg of a product as light orange particles (yield 26.1%).

### Example 3: Synthesis of RGET

Rhodamine 6G (0.024 g, 0.05 mmol) and N,N-diisopropyl ethyl amine (DIPEA) (17 μL, 0.1 mmol) were dissolved in acetonitrile (8 mL). Under a N₂ atmosphere, 2-thiazolylethyl amine (8 μL, 0.075 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 11.7 mg of a product as light orange particles (yield 44.6%).

### Example 4: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (5 μL, 0.05 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 18.4 mg of a product as light orange particles (yield 70.2%).

### Example 5: Synthesis of RGET

Rhodamine 6G (0.024 g, 0.05 mmol) and sodium acetate (CH₃COONa) (0.0082 g, 0.1 mmol) were dissolved in acetonitrile (8 mL), Under a N₂ atmosphere, 2-thiazolylethyl amine (8 μL, 0.075 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 19.1 mg of a product as light orange particles (yield 72.9%).

### Example 6: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (13 μL, 0.125 mmol) was added, and reacted under reflux for 4 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 19.5 mg of a product as light orange particles (yield 74.4%).

### Example 7: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (11 μL, 0.1 mmol) was added, and reacted under reflux for 4 h with heating at 65° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 18.1 mg of a product as light orange particles (yield 69.1%).

### Example 8: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (11 μL, 0.1 mmol) was added, and reacted under reflux for 4 h with heating at 85° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 19.0 mg of a product as light orange particles (yield 72.5%).

### Example 9: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (11 μL, 0.1 mmol) was added, and reacted under reflux for 3 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 17.5 mg of a product as light orange particles (yield 66.8%).

### Example 10: Synthesis of RGET

Rhodamine 6G (0.024 g, 0.05 mmol) and sodium acetate (CH₃COONa) (0.0082 g, 0.1 mmol) were dissolved in acetonitrile (8 mL). Under a N₂ atmosphere, 2-thiazolylethyl amine (11 μL, 0.1 mmol) was added, and reacted under reflux for 6 h with heating at 75° C. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain 19.3 mg of a product as light orange particles (yield 73.7%).

### Example 11. Response of RGET to Metal Ions

To explore the response of RGET to metal ions, three metal ions, Fe³⁺, Al³⁺, and Cr³⁺, to which RGET tended to respond, were used, and the color and fluorescence of an RGET solution in a CH₃CN/H₂O (10/90, 20/80, 30/70, 40/60, 50/50, 60/40, 70/30, 80/20, 90/10, v/v) solvent system before and after the three metal ions added were recorded respectively, where the metal concentration was 50 μM. The result is shown in FIG. 2. RGET is found to respond to Fe³⁺, Al³⁺ and Cr³⁺ (from left to right in each system), and the response to Fe³⁺, Al³⁺, and Cr³⁺ increases with the increase of acetonitrile volume, and shows no selectivity to a certain metal ion.

### Example 12. Response of RGET to Metal Ions

The color, fluorescence, UV-vis absorption spectrum, and fluorescence spectrum of RGET in a DMF/H₂O (1/99, v/v) solvent system before and after Fe³⁺, Fe²⁺, Cr³⁺, Ag⁺, Cu²⁺, Al³⁺, Na⁺, K⁺, Mg²⁺, Ca²⁺, Hg²⁺, Zn²⁺, Co²⁺, Cd²⁺, Pb²⁺, Mn²⁺, or Ni²⁺ was added were further analyzed. The results are shown in FIGS. 3 and 4.

FIG. 3 shows the color under natural light and fluorescence of RGET in DMF/H₂O (1/99, v/v) before and after adding the metal ion, where the metal concentration is 100 μM. It can be seen that only Fe³⁺ causes the RGET solution in DMF/H₂O (1/99, v/v) to turn red, and emit yellow green fluorescence.

FIG. 4 shows the UV-vis absorption spectrum (FIG. 4a) and fluorescence spectrum (FIG. 4b) of an RGET solution in DMF/H₂O (1/99, v/v) before and after the metal ion is added. It can be seen from FIG. 4a that Fe³⁺ causes RGET to have an absorption peak at 531 nm, and the absorbance is enhanced by 43 times. The peak corresponds to the color observed by naked eyes. Other ions do not cause obvious change in the UV-vis absorption spectrum of the RGET solution. It can be seen from FIG. 4b that Fe³⁺ cause the fluorescence of RGET at 554 nm to increase, and the fluorescence intensity is increased by 541 times. Other ions do not obvious change in the fluorescence spectrum of the RGET solution. Therefore, in the DMF/H₂O (1/99, v/v) system, the UV-vis absorption spectrum and fluorescence spectrum of RGET show a highly selective response to Fe³⁺.

### Example 13: Relationship Between Absorption Spectrum and Fluorescence Spectrum of RGET and Fe³⁺ Concentration

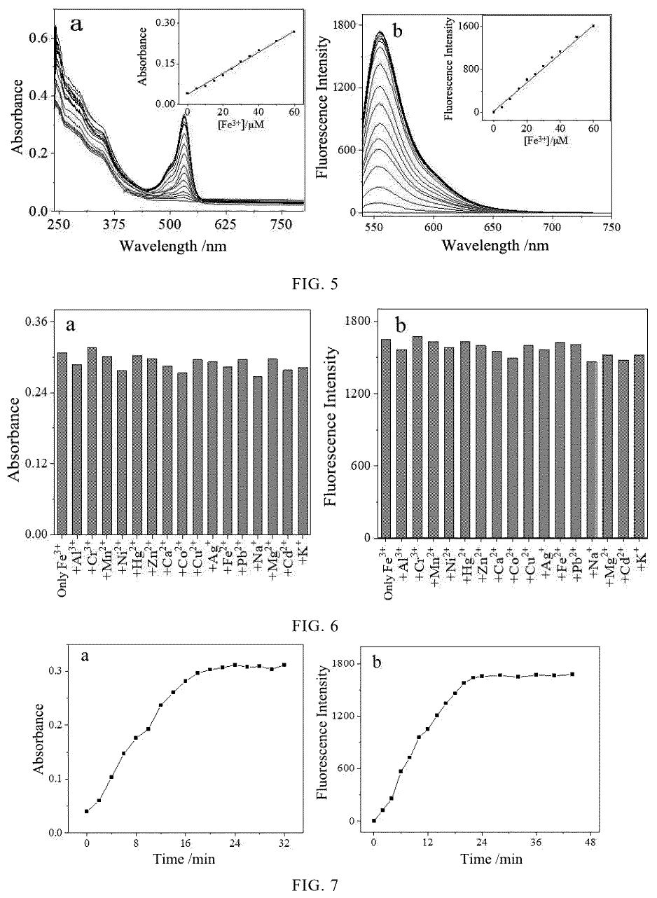

To explore the relationship between the UV-vis absorption spectrum and fluorescence spectrum of RGET and Fe³⁺ concentration, the UV-vis absorption spectrum and fluorescence spectrum of an RGET solution in DMF/H₂O (1/99, v/v) after different concentrations of Fe³⁺ was added were investigated. As shown in FIG. 5, with the gradual increase of Fe³⁺ concentration, the absorbance of RGET at 531 nm (FIG. 5a) and the fluorescence intensity at 554 nm (FIG. 5b) increase gradually. In a concentration range of Fe³⁺ from 0 to 60 μM, and there is a good linear relationship with Fe³⁺ concentration. The linear equations of absorbance and fluorescence intensity are A=0.00395×[Fe³⁺]+0.03449 and F=27.68461×[Fe³⁺]+8.73203, respectively, where the correlation coefficients are 0.997 and 0.997, respectively. The detection limits of Fe³⁺ by UV-vis absorption spectroscopy and fluorescence spectroscopy are 1.4×10⁻⁶ mol/L and 2.1×10−9 mol/L respectively.

### Example 14. Anti-Interference Ability of RGET in Detecting Fe³⁺

To investigate the ability of RGET to withstand the interference from other co-existing ions in a DMF/H₂O (1/99, v/v) system in detecting Fe³⁺, one of the following ions: Al³⁺, Cr³⁺, Fe²⁺, Ag⁺, Cu²⁺, Na⁺, K⁺, Mg²⁺, Ca²⁺, Hg²⁺, Zn²⁺, Co²⁺, Cd²⁺, Pb²⁺, Mn²⁺ and Ni²⁺ was added into the RGET solution containing Fe³⁺. The UV-vis absorption spectra and fluorescence spectra of the solution before and after adding these ions were compared. As shown in FIG. 6, the absorbance at 531 nm (FIG. 6a) and the fluorescence intensity near 554 nm (FIG. 6b) have no obvious change after adding other interfering ions. Therefore, when RGET is used to detect Fe³⁺ in the DMF/H₂O (1/99, v/v) system, it has a high anti-interference ability.

### Example 15. Time Response of RGET in Detecting Fe³⁺

To investigate the response speed of RGET in detecting Fe³⁺ in a DMF/H₂O (1/99, v/v) system, the UV-vis absorption and fluorescence spectra of the solution were recorded from the moment Fe³⁺ is added and at regular intervals, as shown in FIG. 7. It can be seen from FIG. 7a that the absorbance of the RGET solution increases obviously within 16 min after Fe³⁺ is added, and basically reaches a stable state after 20 min. It can be seen from FIG. 7b that the fluorescence intensity of the RGET solution is rapidly increased to a maximum value and stabilized within 22 min after Fe³⁺ is added. Therefore, RGET has the ability to quickly detect Fe³⁺ in the DMF/H₂O (1/99, v/v) system.

### Example 16. Reversibility of RGET in Detecting Fe³⁺

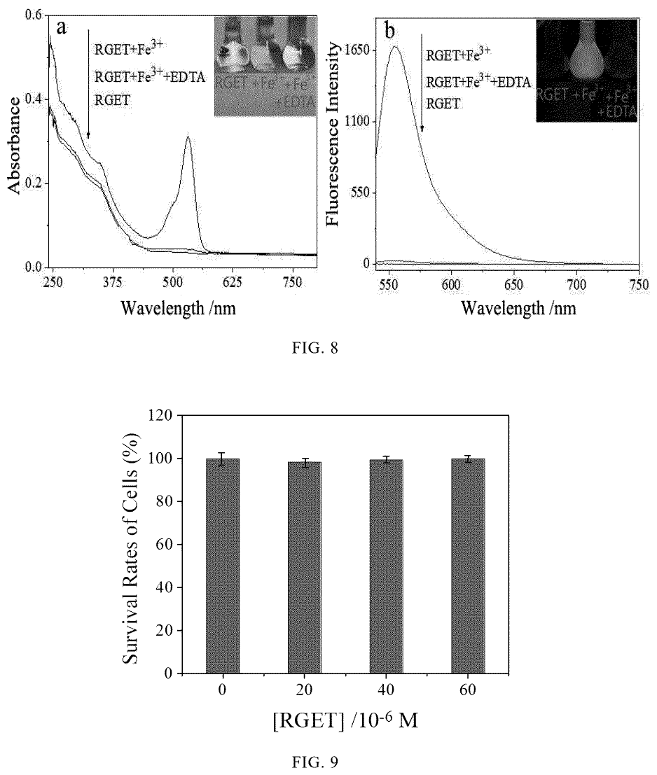

To a solution of RGET in DMF/H₂O (1/99, v/v), Fe³⁺ was added, and then excess EDTA (110 μm) solution was added to investigate the reversibility of RGET in the detection of Fe³⁺. The results are shown in FIG. 8. It can be seen from FIG. 8a that the color of the RGET solution changes from colorless to pink after Fe³⁺ is added, and changes from pink to colorless after EDTA is added. After Fe³⁺ is added to the RGET solution, the absorbance of the solution is analyzed, and an absorption peak is found to appear at 531 nm. The absorbance of the RGET solution decreases to be close to the original absorbance of RGET after EDTA was added. Therefore, RGET has reversibility in colorimetric detection of Fe³⁺. It can be seen from FIG. 8b that after Fe³⁺ is added to the RGET solution, the solution emits yellow fluorescence, and the fluorescence is quenched after the EDTA solution is added. It is found that after Fe³⁺ is added to the RGET solution, the fluorescence intensity of the solution at 554 nm is significantly increased; and after excess EDTA solution is added, the fluorescence intensity at 554 nm is significantly reduced and basically returned to the original fluorescence intensity of RGET. It can be seen that the detection of Fe³⁺ by RGET is a reversible process, which lays a foundation for the recycling of the probe.

### Example 17: Applicability of RGET

To investigate the applicability of RGET, the pond water and tap water from Dushu Lake Campus of Soochow University were subjected to spiked analysis by RGET. The fluorescence emission spectrum of a Fe³⁺ spiked RGET solution containing pond water (or tap water) was determined. The Fe³⁺ concentration, Fe³⁺ recovery rate and relative standard deviation were calculated according to the linear relationship between the fluorescence intensity and Fe³⁺ concentration of the RGET solution. The results are shown in Table 1. The determined Fe³⁺ concentration is close to the added Fe³⁺ concentration, and the recovery rate of Fe³⁺ is in the range of 96.05-102.13%. The relative standard deviation of three parallel experiments is less than 1.09%, so RGET can be used to effectively analyze Fe³⁺ in actual environmental water samples.

TABLE 1
Determination of Fe³⁺ in pond water
and tap water by RGET (n = 3)

| | Fe³⁺ added | Fe³⁺ determined | Recovery | RSD (n = |
|---|---|---|---|---|
| Sample | (10⁻⁶ mol/L) | (10⁻⁶ mol/L) | rate (%) | 3) (%) |
| Tap | 20 | 19.21 | 96.05 | 0.42 |
| water | 30 | 30.29 | 100.96 | 0.89 |
| Pond | 20 | 19.31 | 96.55 | 0.68 |
| water | 30 | 30.64 | 102.13 | 1.09 |

Solvent system: DMF/H₂O (1/99, v/v),
RGET concentration: 10 μM,
RSD: Relative standard deviation

### Example 18. Toxicity of RGET

Cytotoxicity test method: 100 μL cell suspension (about 5000 cells/well) was inoculated into each well of a 96-well plate, and pre-incubated in an incubator at 37° C. and 5% CO₂ for 24 h. Then 10 μL CCK-8 solution was added to each well of the plate. After the culture plate was incubated in the incubator for 1 h, 1 μL RGET solution (the solvent was CH₃CN) of various concentrations was added to the well, and then the culture plate was incubated in the incubator for 24 h. Finally, before reading the plate, the culture plate was gently shaken on a shaker to mix the content, and then the absorbance at 450 nm was read on a microplate reader.

Cytotoxicity test results: The cytotoxicity of RGET measured on the microplate reader is shown in FIG. 9. The survival rate of cells without RGET treatment is 100%, and the survival rates of cells treated with RGET of 20, 40, and 60 μM are 98.47%, 99.36%, and 100.02% respectively, indicating that RGET has no negative effect on the survival rate of Ctenopharyngodon idellus kidney (CIK) cells. Therefore, RGET has very low cytotoxicity and can be certainly used in biological samples.

### Example 19. Antimicrobial Performance of RGET

Antimicrobial performance test method: 16 EP tubes of 1 mL were numbered from 1 to 16. 50 μL of Bacillus thuringiensis standard suspension (Bio-52496) was added to tubes 1-8 respectively, and then 50 μL of sterile water, 50 μL of DMSO solvent, 50 μL of 2.0, 3.0, 3.1, 3.2, and 3.3 mg/mL RGET antibacterial solution (with DMSO as a solvent), and 50 μL of 2.0 mg/mL bleach were respectively added. 50 μL of Serratia marcescens standard suspension (Bio-72774) was added to tubes 9-16 respectively, and other operations were the same as those for tubes 1-8. The system was mixed quickly and the time was recorded immediately. After 30 min interaction of the test bacterium with the antibacterial agent, a sterilized neutralizer was added and mixed well, to stop reaction. After 10 min, 50 μL of evenly mixed reaction solution was added into a Petri dish with the same number and containing LB culture medium. A coating rod was heated and sterilized on the flame of an alcohol lamp, and then dipped in the reaction solution. The reaction solution was evenly coated on the LB culture medium. The culture dish was sealed with a sealing film, placed in a biochemical chamber at 37° C. for natural growth for 24 h, photographed, and recorded.

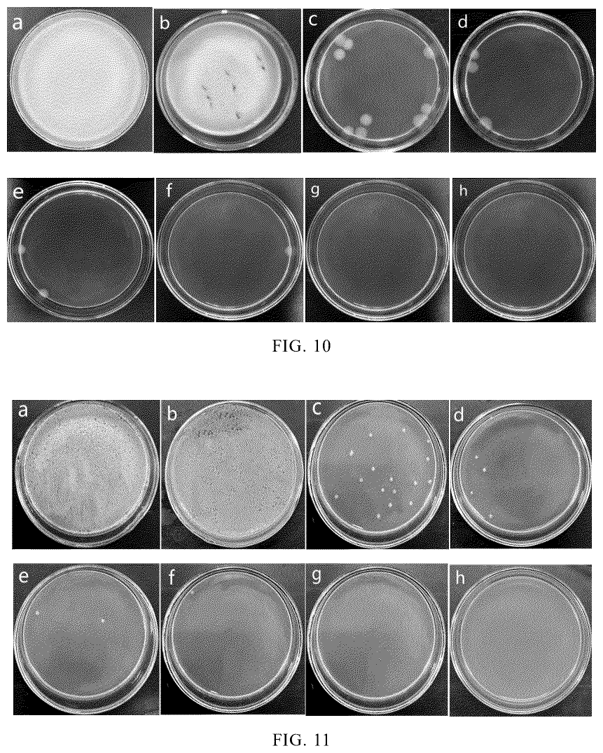

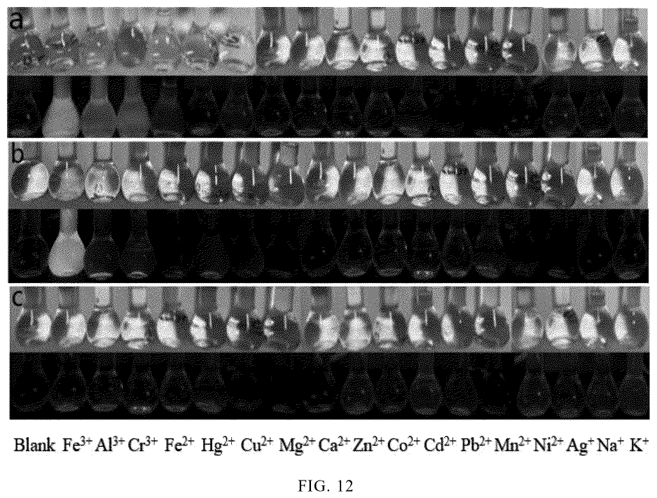

Antibacterial performance test results: The antibacterial performance of RGET is studied by a quantitative killing experiment in suspension. As shown in FIGS. 10 and 11, Bacillus thuringiensis (Gram-positive bacterium, FIG. 10a) and Serratia marcescens (Gram-negative bacterium, FIG. 11a) are grown and propagated all over the surface of the culture medium under suitable growth environment. DMSO as an organic solvent has little effect on the growth of Bacillus thuringiensis (FIG. 10b) and Serratia marcescens (FIG. 11b), and full growth of them on the surface of the medium is also observed, which has almost no obvious difference from the growth in normal environment. By analyzing the effects of various concentrations RGET antibacterial solutions, it can be found that when the concentration of the RGET solution is 2.0 mg/mL, a small number of large white round colonies still grow on the surface of the culture medium with Bacillus thuringiensis (FIG. 10c), and a small number of small white colonies grow on the surface of the culture medium with Serratia marcescens (FIG. 11c), which are slightly different from the bactericidal effect of bleach with the same concentration (FIG. 10h and FIG. 11h). When the concentration of the RGET solution is increased to 3.0 mg/mL (FIG. 10d and FIG. 11d), the bactericidal effect is enhanced (FIGS. 10e to 10f and FIGS. 11e to 11f), but a very small number of colonies still grow. When the concentration of the RGET solution is further increased to 3.3 mg/mL, no colonies are grown on the surface of the culture medium with Bacillus thuringiensis (FIG. 10g) or Serratia marcescens (FIG. 11g), and the bactericidal effect is comparable to that of bleach. It can be seen that RGET can inhibit the growth and reproduction of Gram-positive bacteria and Gram-negative bacteria, and RGET has good antibacterial performance, with the antibacterial effect being similar to that of commercial bleach.

### Comparative Embodiment

As described in Example 1 of Chinese Patent Application No. 202410974311.1, compound RGTM was prepared. The specific steps were as follows. Rhodamine 6G (0.096 g, 0.2 mmol) and sodium acetate (CH₃COONa) (0.033 g, 0.4 mmol) were dissolved in acetonitrile (8 mL), and stirred under a N₂ atmosphere. After 5 min, 2-thiazolylethyl amine (38 μL, 0.4 mmol) was added, heated to 75° C., and reacted under reflux for 7 h with heating. Then the reaction was terminated. The reaction solution was cooled to room temperature, the solvent was removed under reduced pressure, and the crude product was separated by column chromatography on silica gel eluting with ethyl acetate/petroleum ether=1/3 (v/v) to obtain a product as light orange particles. As described in Example 1 of Chinese Patent Application No. 202510168310.2, compound RGAT was prepared. The specific steps were as follows. Rhodamine 6G (0.048 g, 0.1 mmol) and sodium acetate (CH₃COONa) (0.016 g, 0.2 mmol) were dissolved in acetonitrile (8 mL), and stirred normally under a N₂ atmosphere. Then, 2-aminothiazole (0.02 g, 0.2 mmol) was added, and reacted under reflux for 11 h at 75° C. Then the reaction was terminated. The reaction solution was naturally cooled to room temperature, the solvent was removed under reduced pressure, and the crude product was separated by column chromatography on silica gel eluting with ethyl acetate/petroleum ether=1/4 (v/v) to obtain a product as light pink particles.

### Structural Formulas:

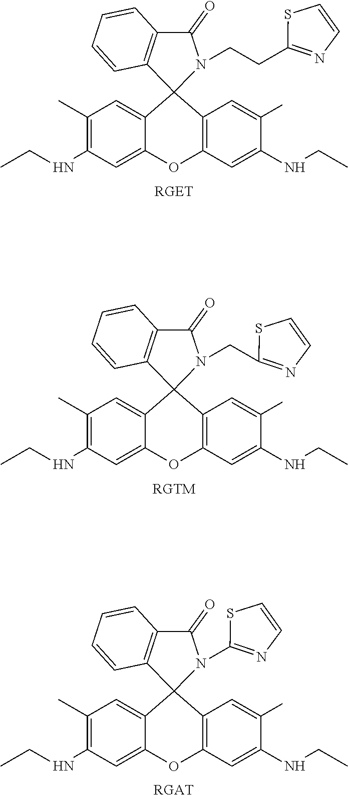

It can be seen that RGET of the present invention is similar to RGTM and RGAT in structure, and RGET has 1 more CH₂ than RGTM and 2 more CH₂ than RGAT. FIG. 12 shows the color under natural light and fluorescence under UV light at 365 nm of 10 UM RGET, RGTM or RGAT solution in DMF/H₂O (1/99, v/v) before and after 50 μM metal ion is added. It can be seen from FIG. 12 that RGET can respond specifically Fe³⁺, with the color change of the solution from colorless to pink and the emission of yellow green fluorescence. RGTM cannot respond specifically to Fe³⁺, but responds to Fe³⁺, Cr³⁺, and Al³⁺ simultaneously. RGAT does not respond to 17 metal ions including Fe³⁺. It can be seen that the slight change of the structure causes an unpredictable and significant difference in the recognition performance.

1-18. (canceled)

19. A multi-functional rhodamine 6G derivative attached with 2-thiazolylethyl amine, having a chemical structural formula of:

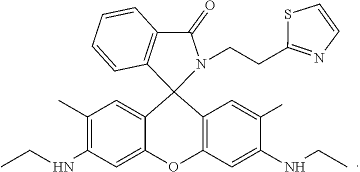 [SMILES: CCNc1cc2c(cc1C)C1(c3cc(C)c(NCC)cc3O2)c2ccccc2C(=O)N1CCc1nccs1]

20. A method for preparing a multi-functional rhodamine 6G derivative attached with 2-thiazolylethyl amine according to claim 19, comprising preparing the multi-functional rhodamine 6G derivative attached with 2-thiazolylethyl amine by a one-step reaction using rhodamine 6G and 2-thiazolylethyl amine as raw materials.

21. The method for preparing a multi-functional rhodamine 6G derivative attached with 2-thiazolylethyl amine according to claim 20, wherein the reaction is carried out under an inert gas atmosphere, the reaction temperature is 50 to 100° C., the reaction time is 1 to 15 h.

22. Use of the multi-functional rhodamine 6G derivative attached with 2-thiazolylethyl amine according to claim 19 as a Fe³⁺ spectroscopic probe for a non-diagnostic or non-treatment purpose of a disease.

23. Use of the multi-functional rhodamine 6G derivative attached with 2-thiazolylethyl amine according to claim 19 in the preparation of a Fe³⁺ detection reagent.

24. Use of the multi-functional rhodamine 6G derivative attached with 2-thiazolylethyl amine according to claim 19 as an antibacterial agent for a non-diagnostic or non-treatment purpose of a disease, or in the preparation of an antibacterial agent.

25. A method for detecting Fe³⁺ for a non-diagnostic or non-treatment purpose of a disease, comprising detecting Fe³⁺ with the multi-functional rhodamine 6G derivative attached with 2-thiazolylethyl amine according to claim 19.

26. The method for detecting Fe³⁺ according to claim 25, wherein the spectroscopic detection comprises ultraviolet-visible (UV-vis) spectroscopic detection and/or fluorescent spectroscopic detection.

27. An antibacterial method for a non-diagnostic or non-treatment purpose of a disease, comprising combating bacteria with the multi-functional rhodamine 6G derivative attached with 2-thiazolylethyl amine according to claim 19.

28. The method according to claim 27, comprising mixing the multi-functional rhodamine 6G derivative attached with 2-thiazolylethyl amine with a bacterium containing system.
